(19) United States
(12) Patent Application Publication
(10) Pub. No.: US 2018/0085214 A1
(43) Pub. Date: Mar. 29, 2018

(54) BUCKLE PAD

(71) Applicant: BOSTON SCIENTIFIC SCIMED, INC., MAPLE GROVE, MN (US)

(72) Inventors: Cornelius M. Crowley, San Francisco, CA (US); Ali Salahieh, Saratoga, CA (US); Takashi H. Ino, San Jose, CA (US); Christopher Blake Finnegan, Los Gatos, CA (US); Floriza Q. Escalona, San Jose, CA (US); Anh Thu Pham, San Jose, CA (US); Bhakta Basnet, Sunnyvale, CA (US); Karl Damon Fleck, Gilroy, CA (US); Leticia Marquez Garcia, Gilroy, CA (US); Jay Yang, Saratoga, CA (US); Brian D. Brandt, Morgan Hill, CA (US); David Chia-Wei Wang, San Jose, CA (US); Connor Gordon Mulcahy, Whitewater, WI (US)

(73) Assignee: BOSTON SCIENTIFIC SCIMED, INC., MAPLE GROVE, MN (US)

(21) Appl. No.: 15/712,260

(22) Filed: Sep. 22, 2017

Related U.S. Application Data
(60) Provisional application No. 62/398,873, filed on Sep. 23, 2016.

(51) Int. Cl. A61F 2/24

(52) U.S. Cl. CPC A61F 2/2409; A61F 2/246; A61F 2220/0008; A61F 2/2427

(57) ABSTRACT

A replacement heart valve implant may include an expandable anchor member actuatable between a delivery configuration and a deployed configuration, a plurality of locking mechanisms configured to maintain the expandable anchor member in the deployed configuration, one or more valve leaflets attached to each of the plurality of locking mechanisms, and at least one polymeric laminate corresponding to each locking mechanism, each of the at least one polymeric laminate sized and configured for mounting between a first portion of the corresponding locking mechanism and the expandable anchor member.

### CROSS REFERENCE TO RELATED APPLICATIONS

This application claims benefit of the provisional U.S. Patent Application No. 62/398,873, filed Sep. 23, 2016, which is hereby incorporated by reference in its entirety.

### TECHNICAL FIELD

The present disclosure pertains to medical devices, and methods for manufacturing and/or using medical devices. More particularly, the present disclosure pertains to a replacement heart valve implant and/or apparatus and methods of manufacture therefor.

### BACKGROUND

A wide variety of intracorporeal medical devices have been developed for medical use, for example, intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

In a first aspect, an apparatus for reducing adhesive wear in a replacement heart valve implant may comprise a polymeric laminate sized and configured for mounting between a first portion of a locking mechanism and an expandable anchor member of the replacement heart valve implant, wherein the first portion of the locking mechanism is non-removably secured to the expandable anchor member.

In addition or alternatively, and in a second aspect, a first surface of the polymeric laminate is parallel to a first surface of the first portion of the locking mechanism.

In addition or alternatively, and in a third aspect, the first surface of the first portion of the locking mechanism faces toward an inner surface of the expandable anchor member.

In addition or alternatively, and in a fourth aspect, the polymeric laminate is at least partially disposed between the first surface of the first portion of the locking mechanism and the inner surface of the expandable anchor member.

In addition or alternatively, and in a fifth aspect, the polymeric laminate abuts the first surface of the first portion of the locking mechanism and the inner surface of the expandable anchor member.

In addition or alternatively, and in a sixth aspect, the first surface of the polymeric laminate is arcuate.

In addition or alternatively, and in a seventh aspect, an outer perimeter of the polymeric laminate extends outward of an outer perimeter of the first surface of the first portion of the locking mechanism.

In addition or alternatively, and in an eighth aspect, the polymeric laminate includes one or more apertures extending through the polymeric laminate.

In addition or alternatively, and in a ninth aspect, the one or more apertures align with one or more corresponding apertures formed in the first portion of the locking mechanism.

In addition or alternatively, and in a tenth aspect, a replacement heart valve implant may comprise an expandable anchor member actuatable between a delivery configuration and a deployed configuration, a plurality of locking mechanisms configured to maintain the expandable anchor member in the deployed configuration, one or more valve leaflets attached to each of the plurality of locking mechanisms, and at least one polymeric laminate corresponding to each locking mechanism, each polymeric laminate sized and configured for mounting between a first portion of the corresponding locking mechanism and the expandable anchor member.

In addition or alternatively, and in an eleventh aspect, each locking mechanism is non-removably secured to the expandable anchor member.

In addition or alternatively, and in a twelfth aspect, actuation of the expandable anchor member between the delivery configuration and the deployed configuration causes relative movement between the expandable anchor member and each locking mechanism.

In addition or alternatively, and in a thirteenth aspect, the expandable anchor member is formed with a braided configuration.

In addition or alternatively, and in a fourteenth aspect, each locking mechanism comprises a buckle member secured to the expandable anchor member and a translatable post member configured to engage the buckle member when the expandable anchor member is in the deployed configuration.

In addition or alternatively, and in a fifteenth aspect, the polymeric laminate prevents contact between the buckle member and the expandable anchor member.

In addition or alternatively, and in a sixteenth aspect, a method of manufacturing an apparatus for reducing adhesive wear in a replacement heart valve implant may comprise stacking a plurality of layers of polyester fabric on a mandrel in a generally flat configuration, spraying the plurality of layers with a polyurethane in the generally flat configuration, wherein the polyurethane impregnates the plurality of layers and adheres the plurality of layers together, laser cutting a perimeter of the apparatus to separate the apparatus from the polyester fabric, and forming the apparatus into a static three-dimensional shape corresponding to an exterior surface of a locking mechanism of the replacement heart valve implant.

In addition or alternatively, and in a seventeenth aspect, laser cutting the perimeter of the apparatus includes laser welding the plurality of layers together along the perimeter.

In addition or alternatively, and in an eighteenth aspect, forming the apparatus includes heat setting the apparatus using a mandrel or die set.

In addition or alternatively, and in a nineteenth aspect, the method may further comprise curing the plurality of layers after spraying the plurality of layers with polyurethane and before laser cutting.

In addition or alternatively, and in a twentieth aspect, the method may further comprise securing the apparatus between the exterior surface of the locking mechanism and an expandable anchor member of the replacement heart valve implant, thereby preventing direct contact between the exterior surface of the locking mechanism and the expandable anchor member.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates an example medical device system;

FIG. 2 illustrates an example replacement heart valve implant;

FIG. 3 is a detailed view of a portion of the example replacement heart valve implant of FIG. 2;

FIG. 4 is a detailed view of a portion of the example replacement heart valve implant of FIG. 2;

FIG. 5 is an exploded view of the portion of the example replacement heart valve implant shown in FIG. 3;

FIG. 6 illustrates a portion of an example method of manufacturing an example apparatus in accordance with the disclosure;

FIG. 7 illustrates a portion of an example method of manufacturing an example apparatus in accordance with the disclosure;

FIG. 8 illustrates an example apparatus in accordance with the disclosure in a flat state; and

FIG. 9 an example apparatus in accordance with the disclosure in a formed state.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g. 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosed invention are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent in the United States and throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians.

Some relatively common medical conditions may include or be the result of inefficiency, ineffectiveness, or complete failure of one or more of the valves within the heart. For example, failure of the aortic valve can have a serious effect on a human and could lead to serious health condition and/or death if not dealt with. Treatment of defective heart valves poses other challenges in that the treatment often requires the repair or outright replacement of the defective valve. Such therapies may be highly invasive to the patient. Disclosed herein are medical devices that may be used within a portion of the cardiovascular system in order to diagnose, treat, and/or repair the system. At least some of the medical devices disclosed herein may include a replacement heart valve (e.g., a replacement aortic valve). In addition, the devices disclosed herein may deliver the replacement heart valve percutaneously and, thus, may be much less invasive to the patient. The devices disclosed herein may also provide a number of additional desirable features and benefits as described in more detail below.

The figures illustrate selected components and/or arrangements of a medical device system 10. It should be noted that in any given figure, some features of the medical device system 10 may not be shown, or may be shown schematically, for simplicity. Additional details regarding some of the components of the medical device system 10 may be illustrated in other figures in greater detail. A medical device system 10 may be used to deliver and/or deploy a variety of medical devices to a number of locations within the anatomy. In at least some embodiments, the medical device system 10 may include a replacement heart valve delivery system (e.g., a replacement aortic valve delivery system) that can be used for percutaneous delivery of a replacement heart valve. This, however, is not intended to be limiting as the medical device system 10 may also be used for other interventions including mitral valve replacement, valve repair, valvuloplasty, and the like, or other similar interventions.

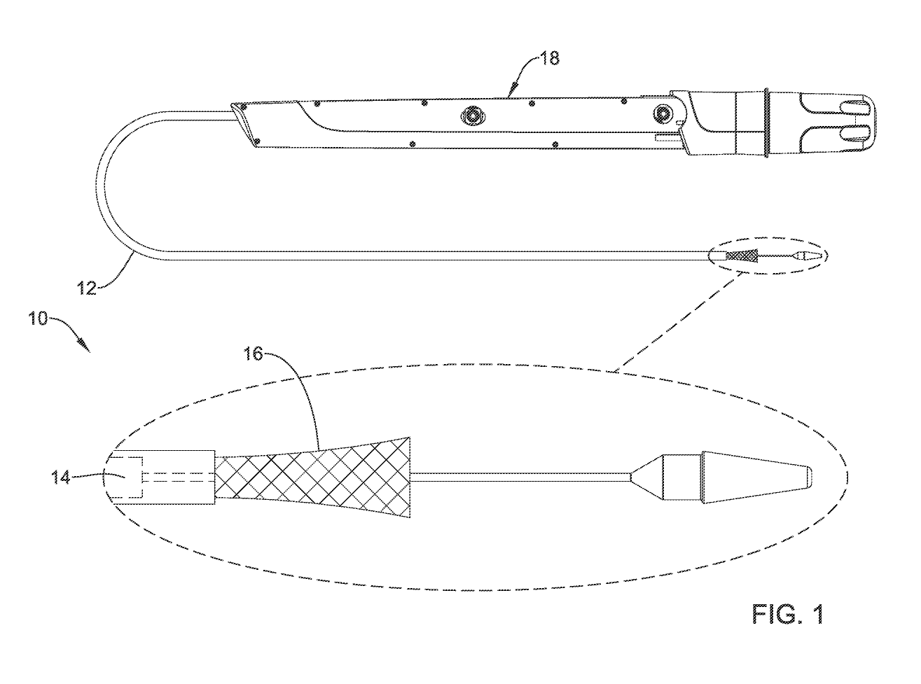

The medical device system 10, as seen in FIG. 1 for example, may generally be described as a catheter system that includes a delivery system having an outer sheath 12 for a medical implant 16 (e.g., a replacement heart valve implant, for example, which term may be used interchangeably with the term “medical implant” herein) which may be coupled to the delivery system and disposed within a lumen of the outer sheath 12 during delivery of the medical implant 16. In some embodiments, the delivery system may include an inner catheter 14 extending at least partially through the outer sheath 12 (partially seen in phantom in FIG. 1). In some embodiments, the medical implant 16 may be coupled to the inner catheter 14 and disposed within the lumen of the outer sheath 12 during delivery of the medical implant 16. In some embodiments, a handle 18 may be disposed and/or attached at a proximal end of the delivery system, as seen in FIG. 1, and may include one or more actuation means associated therewith. In some embodiments, the handle 18 may be configured to manipulate the position of the outer sheath 12 relative to the inner catheter 14, and/or aid in the deployment of the medical implant 16. In some embodiments, the medical device system 10 may include a nose cone disposed at a distal end of a guidewire extension tube, wherein the guidewire extension tube may extend distally from the inner catheter 14. In at least some embodiments, the nose cone may be designed to have an atraumatic shape. In some embodiments, the nose cone may include a ridge or ledge that is configured to abut a distal tip of the outer sheath 12 during delivery of the medical implant 16.

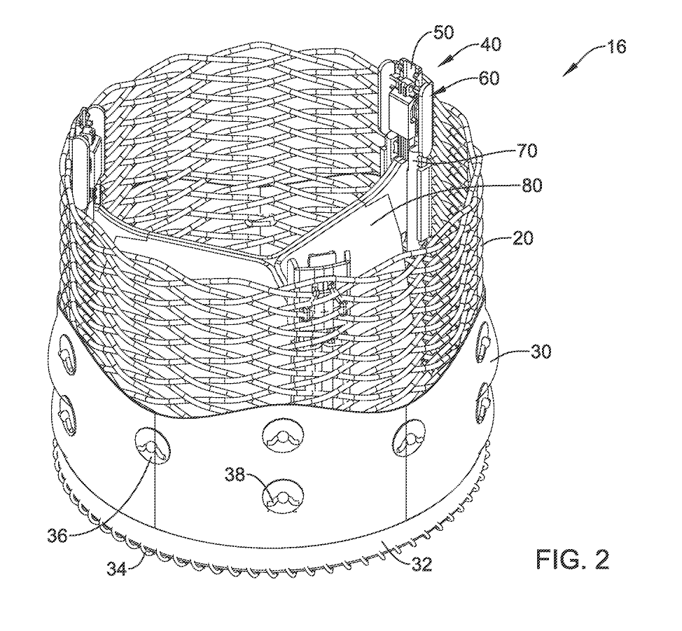

In use, the medical device system 10 may be advanced percutaneously through the vasculature to a position adjacent to an area of interest or a target location. For example, the medical device system 10 may be advanced through the vasculature and across the aortic arch to a position adjacent to a defective aortic valve. Alternative approaches to treat a defective aortic valve and/or other heart valve(s) are also contemplated with the medical device system 10. During delivery, the medical implant 16 may be generally disposed in an elongated and low profile “delivery” configuration within the delivery system and/or the outer sheath 12 coupled to and/or distal of the inner catheter 14. Once positioned, the outer sheath 12 may be retracted relative to the inner catheter 14, which may be held stationary by the handle 18, and/or the medical implant 16 to expose the medical implant 16. The medical implant 16 may be actuated using the handle 18 in order to translate the medical implant 16 into a generally expanded and larger profile “deployed” configuration suitable for implantation within the anatomy (as seen in FIG. 2, for example). When the medical implant 16 is suitably deployed within the anatomy, the medical implant 16 may be released and/or detached from the medical device system 10, the delivery system can be removed from the vasculature, leaving the medical implant 16 in place in a “released” configuration to function as, for example, a suitable replacement for the native aortic valve. In at least some interventions, the medical implant 16 may be deployed within the native valve (e.g., the native valve is left in place and not excised). Alternatively, the native valve may be removed (such as through valvuloplasty, for example) and the medical implant 16 may be deployed in its place as a replacement.

In some embodiments, the inner catheter 14 may include one or more lumens extending therethrough. For example, in some embodiments, the inner catheter 14 may include a first lumen, a second lumen, a third lumen, and a fourth lumen. Other configurations are also contemplated. In general, the one or more lumens extend along an entire length of the inner catheter 14. Other embodiments are contemplated, however, where one or more of the one or more lumens extend along only a portion of the length of the inner catheter 14. In some embodiments, a distal region of the inner catheter 14 may include a step in outer diameter that defines a decreased diameter section. In some embodiments, the decreased diameter section may define a region where other components of the medical device system 10 may be attached. For example, in some embodiments, a coupler assembly may be attached to the inner catheter 14 at the decreased diameter section and/or at a distal end of the inner catheter 14. In some embodiments, the coupler assembly may releasably couple the medical implant 16 to the inner catheter 14.

In some embodiments, disposed within one of the lumens of the inner catheter 14 may be at least one actuator element, which may be used to actuate (e.g., translate axially or longitudinally, and/or expand) the medical implant 16 between a delivery configuration and a deployed configuration. In some embodiments, the medical device system 10 may include at least one actuator element. In some embodiments, the at least one actuator element may include a plurality of actuator elements, two actuator elements, three actuator elements, four actuator elements, or another suitable or desired number of actuator elements. For the purpose of illustration only, the medical device system 10 and/or the medical implant 16 of FIG. 2 is configured to use three actuator elements (not shown). In use, a proximal end of an actuator element may be connected to the handle 18, and/or manipulated or otherwise actuated by a user using the handle 18, to shift the expandable anchor member 20 and/or the medical implant 16 from a “delivery” configuration to a “deployed” configuration, and later to a “released” configuration. During the release process for the medical implant 16, (e.g., as the medical implant 16 is actuated from the “delivery” configuration to the “deployed” configuration to the “released” configuration), the at least one actuator element may be retracted, withdrawn, and/or translated proximally relative to the inner catheter 14, the medical implant 16, and/or the expandable anchor member 20. Some suitable but non-limiting materials for the actuator element, for example metallic materials or polymeric materials, may be described below.

It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the actuator element”, “the locking element”, “the lumen”, or other features may be equally referred to all instances and quantities beyond one of said feature. As such, it will be understood that the following discussion may apply equally to any and/or all of the components for which there are more than one within the medical implant 16 (e.g., the at least one actuator element, the plurality of locking elements, etc.) and/or the medical device system 10, unless explicitly stated to the contrary. Additionally, not all instances of some elements or features may be shown in each figure for clarity.

FIGS. 2-5 illustrate selected components of the medical implant 16. For example, the medical implant 16 may include a plurality of valve leaflets 80 (e.g., bovine pericardial, polymeric, etc.) which may be secured to an expandable anchor member 20 that is reversibly actuatable between an elongated “delivery” configuration (as seen in FIG. 1), and an expanded “deployed” configuration. In some embodiments, the expandable anchor member 20 may form a tubular structure defining a central longitudinal axis and a lumen extending through the expandable anchor member 20 along, parallel to, coaxial with, and/or coincident with the central longitudinal axis. In some embodiments, the expandable anchor member 20 may be formed with and/or may include a braid and/or a braided configuration formed from one or more filaments or wires (e.g., a single filament or wire, two filaments or wires, etc.). Other configurations are also contemplated. Some suitable but non-limiting materials for the expandable anchor member 20, for example metallic materials or polymeric materials, are described below. In some embodiments, the expandable anchor member 20 may form a plurality of filament intersections, where individual filaments or filament segments cross over or under each other, distributed around a circumference of the expandable anchor member 20.

In some embodiments, the medical implant 16 may include a plurality of locking mechanisms 40 attached to the expandable anchor member 20, the plurality of locking mechanisms 40 being configured to reversibly lock the expandable anchor member 20 in the “deployed” and/or “released” configuration(s). In some embodiments, at least one actuator element may be configured to actuate the expandable anchor member 20 and/or the medical implant 16 between the “delivery” configuration and the “deployed” configuration and/or the “released” configuration.

In some embodiments, the plurality of locking mechanisms 40 may each comprise an axially movable post member 70, for example at the commissure portions of the valve leaflets 80 (the post member 70 may sometimes be referred to as a portion of a commissure post, which may serve to secure the valve leaflets 80, or the post member 70 may be connected and/or attached to a commissure post), and a buckle member 50 or other receiving element configured to slidably receive the post member 70 therein to engage with the buckle member 50 and thereafter lock the expandable anchor member 20 and/or the medical implant 16 in the “deployed” and/or the “released” configuration(s). In other words, in at least some embodiments, a medical implant 16 may include a plurality of post members 70 and a corresponding plurality of buckle members 50. Other configurations and correspondences are also contemplated. Some suitable but non-limiting materials for the buckle member 50 and/or the post member 70, for example metallic materials or polymeric materials, are described below.

In some embodiments, the plurality of valve leaflets 80 may be secured to the expandable anchor member 20 at, adjacent to, and/or using (at least in part) individual, corresponding post members 70. In some embodiments, the plurality of valve leaflets 80 may also be secured to a distal end of the expandable anchor member 20. In at least some embodiments, a distal end of the expandable anchor member 20 may be interchangeably described as an “inflow” end or an “upstream” end of the expandable anchor member 20 and/or the medical implant 16. In some embodiments, the plurality of valve leaflets 80 may be coupled and/or secured (e.g., to the post member 70, to the expandable anchor member 20, and/or back to themselves) using one or more sutures, threads, wires, filaments, or other suitable elements. In some embodiments, the plurality of valve leaflets 80 may be coupled and/or secured (e.g., to the post member 70, to the expandable anchor member 20, and/or back to themselves) using an adhesive, a bonding agent, or other suitable securing means. In some embodiments, the plurality of valve leaflets 80 may be coupled and/or secured (e.g., to the post member 70, to the expandable anchor member 20, and/or back to themselves) using a fabric strip, a textile, or other thin flexible material. In some embodiments, the plurality of valve leaflets 80 may not be directly attached to the expandable anchor member 20.

In some embodiments, the post members 70 and/or the commissure posts may be secured and/or attached to the expandable anchor member 20 (e.g., along the interior of the expandable anchor member) with sutures, tethers, adhesives, or other suitable elements. In some embodiments, the commissure post and/or the post member 70 may include one or more holes or other features provided to aid in securing and/or attaching the commissure post and/or the post member 70 to the expandable anchor member 20. Positioned adjacent to (e.g., aligned with) the plurality of post members 70 are a corresponding plurality of buckle members 50, which may be non-removably secured and/or fixedly attached to the expandable anchor member 20 (e.g., along an inner surface of the expandable anchor member 20) with sutures, adhesives, or other suitable mechanisms. In some embodiments, the post member 70 may be axially translatable relative to the buckle member 50 generally parallel to the central longitudinal axis of the expandable anchor member 20 when the post member 70 is at least partially disposed within and/or engaged with the buckle member 50.

In some embodiments, one buckle member 50 may be fixedly attached to the expandable anchor member 20 adjacent to each of the three post members 70. Accordingly, in some embodiments, the expandable anchor member 20 may have a total of three buckle members 50 and three post members 70 attached thereto. Similarly, one actuator element may be associated with each post member 70 and buckle member 50, for a total of three actuator elements in the illustrated example(s). Other embodiments are contemplated where fewer or more buckle members 50, post members 70, and/or actuator elements may be utilized.

In some embodiments, a seal member 30 may be circumferentially disposed on and/or about a distal portion of the expandable anchor member 20, as seen in FIG. 2 for example, and as the term suggests, may help to seal an exterior of the medical implant 16 within and/or against a target site or area of interest upon deployment, thereby preventing leakage around the medical implant 16. In some embodiments, the seal member 30 may be disposed about the expandable anchor member 20. In some embodiments, the seal member 30 may be disposed around a perimeter and/or on or against an exterior surface of the expandable anchor member 20. In some embodiments, the seal member 30 may be coupled and/or secured to the expandable anchor member 20.

In some embodiments, the seal member 30 may include a plurality of layers of polymeric material. Some suitable polymeric materials may include, but are not necessarily limited to, polycarbonate, polyurethane, polyamide, polyether block amide, polyethylene, polyethylene terephthalate, polypropylene, polyvinylchloride, polytetrafluoroethylene, polysulfone, and copolymers, blends, mixtures or combinations thereof. Other configurations and/or other suitable materials are also contemplated.

In some embodiments, the modulus of elasticity may vary and/or be different from layer to layer. In other embodiments, the elongation to break may vary and/or be different from layer to layer. In some embodiments, the seal member 30 may also include a reinforcement, a reinforcing layer, and/or one or more reinforcing members added to the polymeric material prior to curing. The reinforcement, the reinforcing layer, and/or the one or more reinforcing members may comprise a woven or nonwoven fabric and may be positioned within or between the various layers. In some embodiments, the reinforcement, the reinforcing layer, and/or the one or more reinforcing members may be positioned on a radially innermost surface or radially outermost surface of the seal member 30. In some embodiments, the reinforcement, the reinforcing layer, and/or the one or more reinforcing members may be generally aligned. In some embodiments, the reinforcement, the reinforcing layer, and/or the one or more reinforcing members may be randomly oriented and/or disposed on the seal member 30.

In some embodiments, a distal end of the seal member 30 may include a reinforcing band 32 coupled to the seal member 30. In some embodiments, the reinforcing band 32 may be integrally formed with, incorporated into, adhered to, and/or at least partially embedded within the seal member 30. In some embodiments, the reinforcing band 32 may be formed from a woven or nonwoven fabric strip, a textile, or other thin flexible material. The reinforcing band 32 may provide tear resistance in the vicinity of sutures, filaments, or other attachment elements associated with components or aspects of the medical implant 16.

In some embodiments, a distal end of each one of the plurality of valve leaflets 80 may be secured directly to the reinforcing band 32 and/or a distal end of the reinforcing band 32. In some embodiments, the plurality of valve leaflets 80 may not be secured directly to the distal end of the expandable anchor member 20. In some embodiments, the reinforcing band 32 may include a plurality of perforations extending through the reinforcing band 32 and/or the seal member 30. In some embodiments, the plurality of perforations may accommodate one or more sutures 34 passing therethrough (e.g., through the reinforcing band 32 and/or the seal member 30) to secure elements or aspects of the medical implant 16, such as (but not limited to) the plurality of valve leaflets 80 and/or the expandable anchor member 20, for example. In some embodiments, the medical implant 16 may include a plurality of grommets 36 (or other securing members) configured to secure the seal member 30 to the expandable anchor member 20. In some embodiments, each of the plurality of grommets 36 may be secured to the expandable anchor member 20 using individual sutures 38, adhesives, welding, etc. or other suitable means of securement.

It has been found that during manufacture of the medical implant 16, such as during attachment of the buckle member 50 to the expandable anchor member 20, during sheathing of the medical implant 16 within the outer sheath 12 in the delivery configuration, and/or during actuation of the expandable anchor member 20 between the delivery configuration and the deployed configuration, relative movement between the buckle member 50 of each locking mechanism 40 and the expandable anchor member 20 may occur. Such relative movement may contribute to mechanical abrasion, adhesive wear, and/or “smear” between contacting surfaces of the buckle member 50 and the expandable anchor member 20, and/or of these components.

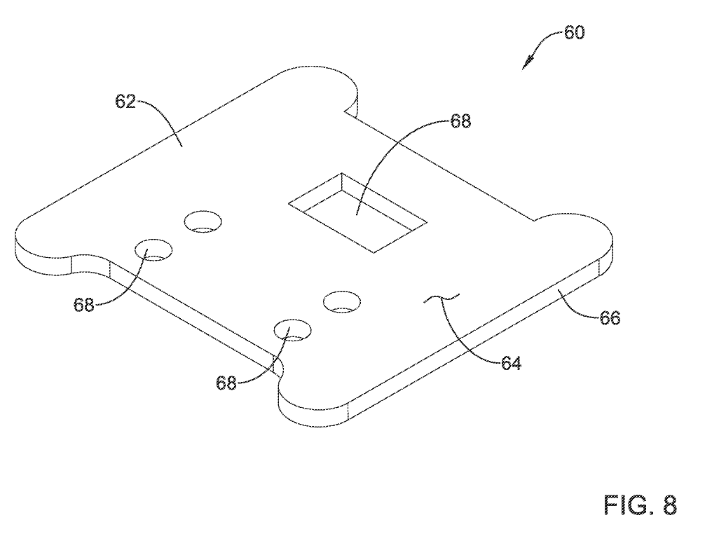

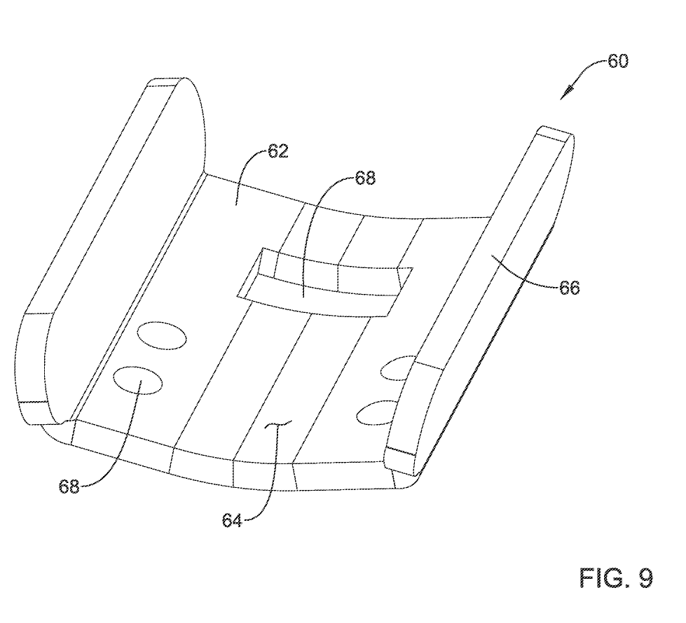

In some embodiments, the medical implant 16 may include an apparatus for reducing mechanical abrasion, adhesive wear, and/or “smear” between the buckle member 50 and the expandable anchor member 20. In some embodiments, the medical implant 16 may include one apparatus for reducing mechanical abrasion, adhesive wear, and/or “smear” corresponding to each of the plurality of locking mechanisms 40. While discussed here with respect to FIGS. 2-5 for context, some features and/or constructional details of the apparatus may be better seen in FIGS. 8 and 9. In some embodiments, the apparatus for reducing mechanical abrasion, adhesive wear, and/or “smear” may be a buckle pad 60 comprising a polymeric laminate 62 sized and configured for mounting between a first portion of one of the plurality of locking mechanisms 40 (e.g., the buckle member 50) and an inner surface of the expandable anchor member 20.

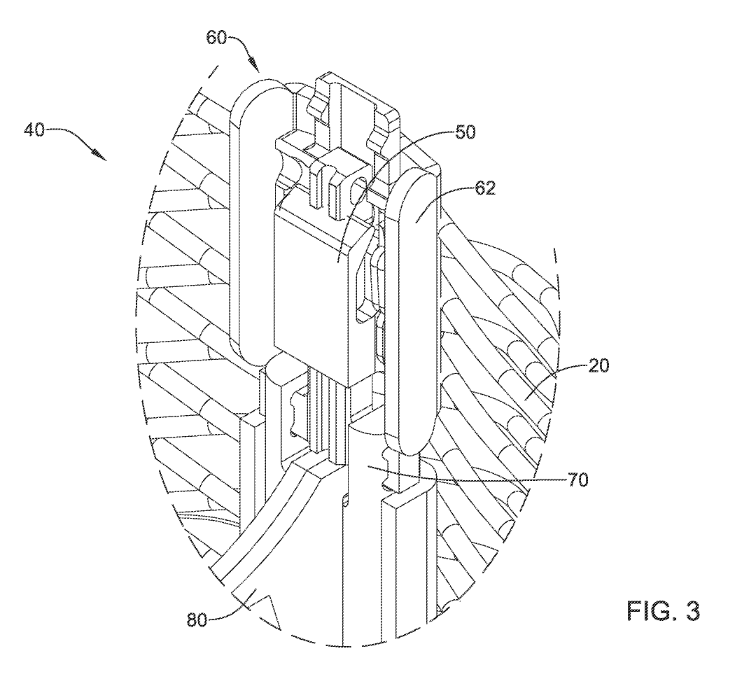

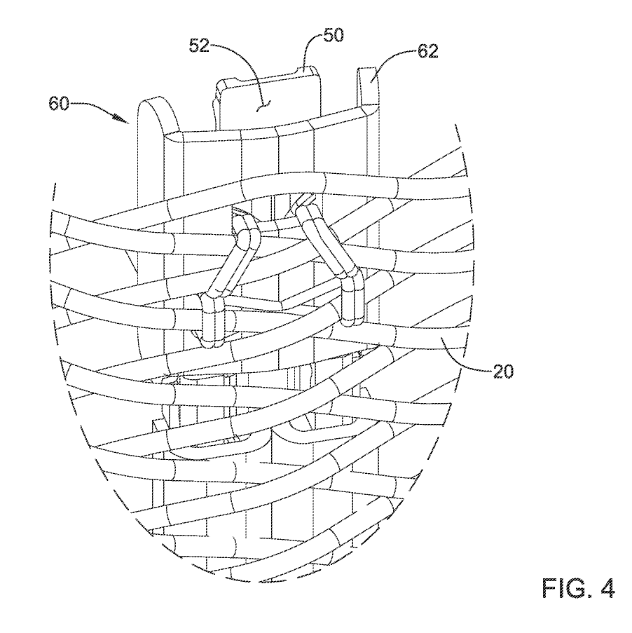

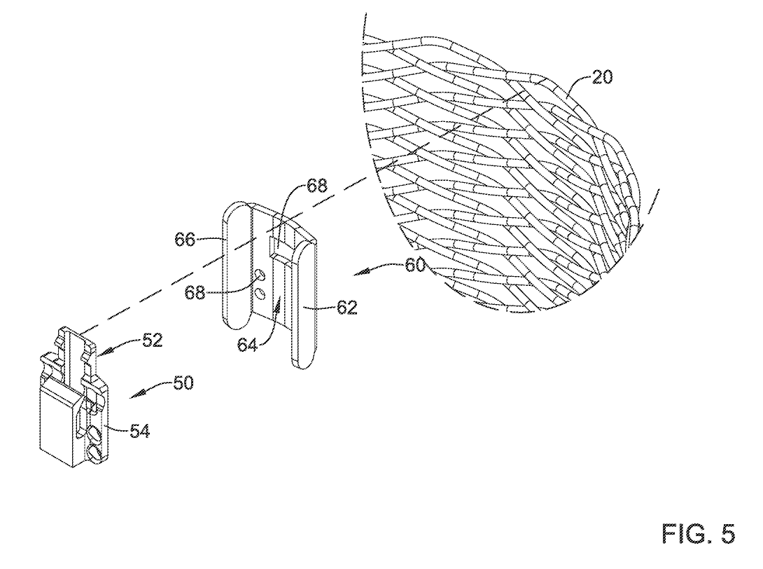

In some embodiments, the polymeric laminate 62 of the buckle pad 60 may be at least partially disposed between a first surface 52 (e.g., an outward-facing surface) of the first portion of one of the plurality of locking mechanisms 40 (e.g., the buckle member 50) and the inner surface of the expandable anchor member 20, as shown in FIG. 3 for example. In some embodiments, the first surface 52 (e.g., an outward-facing surface) of the first portion of one of the plurality of locking mechanisms 40 (e.g., the buckle member 50) is configured and/or oriented to face toward the inner surface of the expandable anchor member 20, as shown in FIG. 4 for example. In some embodiments, a first surface 64 (e.g., an inner-facing surface) of the polymeric laminate 62 of the buckle pad 60 may be oriented substantially parallel to the first surface 52 (e.g., an outward-facing surface) of the first portion of one of the plurality of locking mechanisms 40 (e.g., the buckle member 50), as seen in FIG. 5 for example. In some embodiments, the polymeric laminate 62 of the buckle pad 60 abuts the first surface 52 (e.g., an outward-facing surface) of the first portion of one of the plurality of locking mechanisms 40 (e.g., the buckle member 50) and the inner surface of the expandable anchor member 20.

In some embodiments, the first surface 64 (e.g., an inner-facing surface) of the polymeric laminate 62 of the buckle pad 60 may be arcuate. In some embodiments, a curvature of the first surface 64 (e.g., an inner-facing surface) of the polymeric laminate 62 of the buckle pad 60 may correspond to and/or align with a curvature of the first surface 52 (e.g., an outward-facing surface) of the first portion of one of the plurality of locking mechanisms 40 (e.g., the buckle member 50). In some embodiments, an outer perimeter 66 of the polymeric laminate 62 of the buckle pad 60 may extend outward of and/or beyond an outer perimeter 54 of the first surface 52 (e.g., an outward-facing surface) of the first portion of one of the plurality of locking mechanisms 40 (e.g., the buckle member 50). In some embodiments, the polymeric laminate 62 of the buckle pad 60 may prevent contact between the first surface 52 (e.g., an outward-facing surface) of the first portion of one of the plurality of locking mechanisms 40 (e.g., the buckle member 50) and the inner surface of the expandable anchor member 20.

In some embodiments, the polymeric laminate 62 of the buckle pad 60 may include one or more apertures 68 extending through the polymeric laminate 62 of the buckle pad 60. In some embodiments, the one or more apertures 68 may align with one or more corresponding apertures formed in the first portion of one of the plurality of locking mechanisms 40 (e.g., the buckle member 50). In some embodiments, the one or more apertures 68 may facilitate securement of the apparatus for reducing mechanical abrasion, adhesive wear, and/or “smear” (e.g., the buckle pad 60 and/or the polymeric laminate 62) between and/or to the expandable anchor member 20 and/or the first portion of one of the plurality of locking mechanisms 40 (e.g., the buckle member 50), using the sutures, adhesives, or other suitable mechanisms used to secure the buckle member 50 to the expandable anchor member 20.

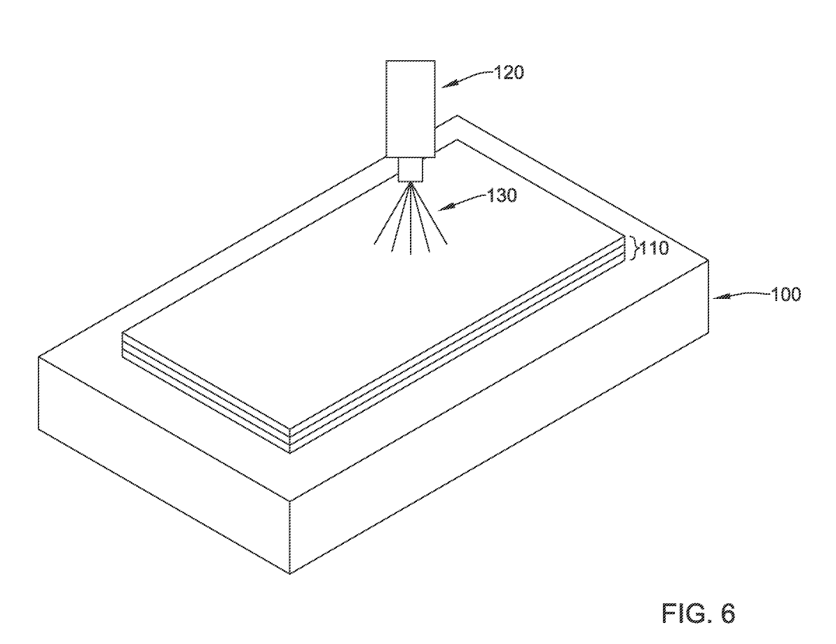

Turning to FIG. 6, a method of manufacturing the apparatus for reducing mechanical abrasion, adhesive wear, and/or “smear” (e.g., the buckle pad 60 and/or the polymeric laminate 62) in a medical implant 16 (e.g., a replacement heart valve implant) may include stacking a plurality of layers 110 of biocompatible fabric (e.g., polyester fabric, etc.) on a mandrel 100 or other suitable platform in a generally flat configuration and/or a generally flat state. In some embodiments, stacking the plurality of layers 110 of biocompatible fabric on the mandrel 100 may include positioning each layer one on top of another on the mandrel 100 in sequence, may include positioning multiple layers together onto the mandrel 100 at a time, and/or may include sandwiching multiple layers together to form a single stack, wherein the single stack is positioned on the mandrel 100. While illustrated in FIG. 6 as three layers, the plurality of layers 110 may include other suitable quantities of layers including, but not limited to, two layers, four layers, five layers, etc. as appropriate for the intended use. In some embodiments, the plurality of layers 110 may include a bottom layer, a top layer, and at least one middle layer positioned between the bottom layer and the top layer. In at least some embodiments, the at least one middle layer may separate the bottom layer and the top layer, thereby preventing direct contact between the bottom layer and the top layer. Other configurations and/or arrangements are also contemplated. For example, in some embodiments, the at least one middle layer may be discontinuous, perforated, and/or include openings permitting contact and/or bonding between the bottom layer and the top layer. In some embodiments, the at least one middle layer may not extend all the way to the perimeter of the bottom layer and/or the top layer such that the bottom layer and/or the top layer surround at least a portion of the perimeter the at least one middle layer, and therefore at least a portion of the bottom layer and the top layer may be in direct contact with each other.

In some embodiments, a spray head 120 may be movably positioned in proximity to the plurality of layers 110 above the mandrel 100 and/or the plurality of layers 110. The spray head 120 may be fluidly connected to a matrix source, such as a liquid polymeric material (including but not limited to polyurethane, etc.). In some embodiments, the liquid polymeric material may be the same material as that used for the seal member 30, discussed above. Some other non-limiting examples of suitable polymeric materials are listed below. In some embodiments, the method may include spraying the plurality of layers 110 with a liquid polymeric material 130 (e.g., polyurethane, etc.) in the generally flat configuration or flat state. The liquid polymeric material 130 may permeate, impregnate, and/or saturate the plurality of layers 110 and adhere the plurality of layers 110 together. In some embodiments, the method may further include curing the plurality of layers 110 after spraying the plurality of layers 110 with the liquid polymeric material 130.

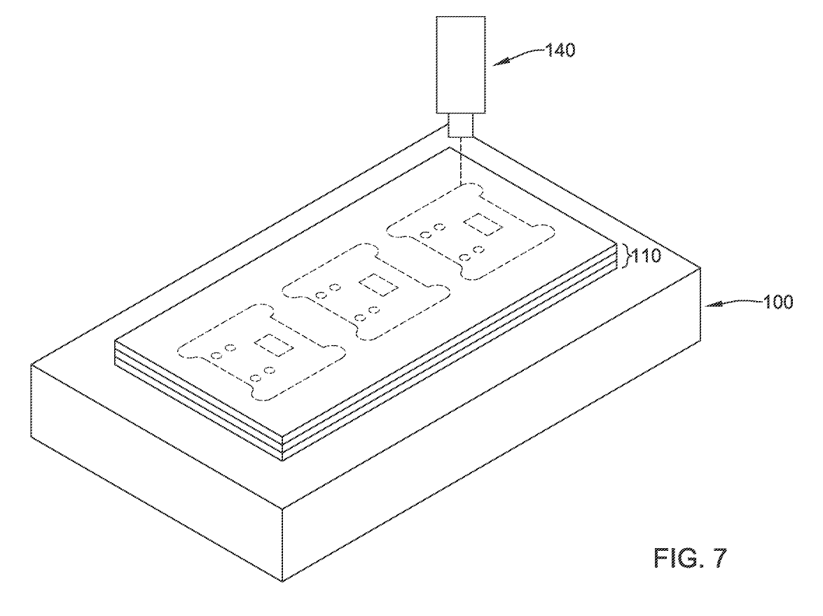

As may be seen in FIG. 7, a laser head 140 may be movably positioned in proximity to the plurality of layers 110 above the mandrel 100 and/or the plurality of layers 110. In some embodiments, the spray head 120 may be replaced and/or interchanged with the laser head 140. In some embodiments, a single machine apparatus may include both the spray head 120 and the laser head 140, wherein each of the spray head 120 and the laser head 140 are capable of being positioned in proximity to the mandrel 100. In some embodiments, the plurality of layers 110 may be transferred from the mandrel 100 to a second mandrel associated with the laser head 140. In some embodiments, the plurality of layers 110 may be transferred between a first machine apparatus including the spray head 120 and a second machine apparatus including the laser head 140. In some embodiments, the mandrel 100 may be transferred between a first machine apparatus including the spray head 120 and a second machine apparatus including the laser head 140 with the plurality of layers 110 still positioned thereon. Other configurations are also contemplated. In some embodiments, the method may include laser cutting the outer perimeter 66 of the apparatus for reducing mechanical abrasion, adhesive wear, and/or “smear” (e.g., the buckle pad 60 and/or the polymeric laminate 62) within the plurality of layers 110 to separate the apparatus for reducing mechanical abrasion, adhesive wear, and/or “smear” (e.g., the buckle pad 60 and/or the polymeric laminate 62) from the permeated, impregnated, and/or saturated plurality of layers 110 of polyester fabric with a desired size, shape, etc. The outer perimeter 66 may represent and/or correspond to a laser cutting path illustrated on an upper surface of the plurality of layers 110 in FIG. 7. In some embodiments, laser cutting the outer perimeter 66 may include laser cutting the one or more apertures 68

In some embodiments, laser cutting the outer perimeter 66 of the apparatus for reducing mechanical abrasion, adhesive wear, and/or “smear” (e.g., the buckle pad 60 and/or the polymeric laminate 62) may include and/or cause laser welding the plurality of layers 110 together along the outer perimeter 66 of the apparatus for reducing mechanical abrasion, adhesive wear, and/or “smear” (e.g., the buckle pad 60 and/or the polymeric laminate 62). A flat state of the apparatus for reducing mechanical abrasion, adhesive wear, and/or “smear” (e.g., the buckle pad 60 and/or the polymeric laminate 62), after laser cutting from the plurality of layers 110, may be seen in FIG. 8.

In some embodiments, the method may include forming the apparatus for reducing mechanical abrasion, adhesive wear, and/or “smear” (e.g., the buckle pad 60 and/or the polymeric laminate 62) from the flat state into a three-dimensional static shape or formed state, as seen in FIG. 9, corresponding to an exterior surface and/or the first surface 52 (e.g., an outward-facing surface) of the first portion of one of the plurality of locking mechanisms 40 (e.g., the buckle member 50) of the medical implant 16 (e.g., the replacement heart valve implant). In some embodiments, forming the apparatus for reducing mechanical abrasion, adhesive wear, and/or “smear” (e.g., the buckle pad 60 and/or the polymeric laminate 62) from the flat state into the three-dimensional static shape or formed state may include heat setting the apparatus using a mandrel or die set.

In some embodiments, the method may further include securing the apparatus for reducing mechanical abrasion, adhesive wear, and/or “smear” (e.g., the buckle pad 60 and/or the polymeric laminate 62) between the exterior surface and/or the first surface 52 (e.g., an outward-facing surface) of the first portion of one of the plurality of locking mechanisms 40 (e.g., the buckle member 50) and the expandable anchor member 20 of the medical implant 16 (e.g., the replacement heart valve implant), thereby preventing direct contact between the exterior surface and/or the first surface 52 (e.g., an outward-facing surface) of the first portion of one of the plurality of locking mechanisms 40 (e.g., the buckle member 50) and the expandable anchor member 20.

It is also contemplated that in some embodiments, the apparatus for reducing mechanical abrasion, adhesive wear, and/or “smear” (e.g., the buckle pad 60 and/or the polymeric laminate 62) may be made by pre-cutting and positioning individual layers of the plurality of layers 110 on a mandrel or die set defining the three-dimensional static shape or formed state before spraying and/or curing. The plurality of layers 110 may then be sprayed with a liquid polymeric material and cured, as discussed above, directly into the three-dimensional static shape or formed state. As such, it may be possible for the apparatus for reducing mechanical abrasion, adhesive wear, and/or “smear” (e.g., the buckle pad 60 and/or the polymeric laminate 62) to be made using fewer steps, tools, mandrels, machines, etc.

The materials that can be used for the various components of the medical device system 10, the outer sheath 12, the inner catheter 14, the medical implant 16, the expandable anchor member 20, the locking mechanisms 40, the buckle pad 60, etc. (and/or other systems disclosed herein) and the various elements thereof disclosed herein may include those commonly associated with medical devices. For simplicity purposes, the following discussion makes reference to the medical device system 10, the outer sheath 12, the inner catheter 14, the medical implant 16, the expandable anchor member 20, the locking mechanisms 40, the buckle pad 60, etc. However, this is not intended to limit the devices and methods described herein, as the discussion may be applied to other elements, members, components, or devices disclosed herein, such as, but not limited to, the grommets 36, the buckle members 50, the polymeric laminate 62, the post members 70, etc. and/or elements or components thereof.

In some embodiments, the medical device system 10, the outer sheath 12, the inner catheter 14, the medical implant 16, the expandable anchor member 20, the locking mechanisms 40, the buckle pad 60, etc., and/or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the medical device system 10, the outer sheath 12, the inner catheter 14, the medical implant 16, the expandable anchor member 20, the locking mechanisms 40, the buckle pad 60, etc., and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the medical device system 10, the outer sheath 12, the inner catheter 14, the medical implant 16, the expandable anchor member 20, the locking mechanisms 40, the buckle pad 60, etc. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device system 10, the outer sheath 12, the inner catheter 14, the medical implant 16, the expandable anchor member 20, the locking mechanisms 40, the buckle pad 60, etc. to achieve the same result.

In some embodiments, a degree of Magnetic Resonance Imaging (MM) compatibility is imparted into the medical device system 10, the outer sheath 12, the inner catheter 14, the medical implant 16, the expandable anchor member 20, the locking mechanisms 40, the buckle pad 60, etc. For example, the medical device system 10, the outer sheath 12, the inner catheter 14, the medical implant 16, the expandable anchor member 20, the locking mechanisms 40, the buckle pad 60, etc., and/or components or portions thereof, may be made of a material that does not substantially distort the image and create substantial artifacts (e.g., gaps in the image). Certain ferromagnetic materials, for example, may not be suitable because they may create artifacts in an MM image. The medical device system 10, the outer sheath 12, the inner catheter 14, the medical implant 16, the expandable anchor member 20, the locking mechanisms 40, the buckle pad 60, etc., or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-N® and the like), nitinol, and the like, and others.

In some embodiments, the medical device system 10, the outer sheath 12, the inner catheter 14, the medical implant 16, the expandable anchor member 20, the locking mechanisms 40, the buckle pad 60, etc., and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. An apparatus for reducing adhesive wear in a replacement heart valve implant, comprising:
a polymeric laminate sized and configured for mounting between a first portion of a locking mechanism and an expandable anchor member of the replacement heart valve implant;
wherein the first portion of the locking mechanism is non-removably secured to the expandable anchor member.

2. The apparatus of claim 1, wherein a first surface of the polymeric laminate is parallel to a first surface of the first portion of the locking mechanism.

3. The apparatus of claim 1, wherein the first surface of the first portion of the locking mechanism faces toward an inner surface of the expandable anchor member.

4. The apparatus of claim 3, wherein the polymeric laminate is at least partially disposed between the first surface of the first portion of the locking mechanism and the inner surface of the expandable anchor member.

5. The apparatus of claim 4, wherein the polymeric laminate abuts the first surface of the first portion of the locking mechanism and the inner surface of the expandable anchor member.

6. The apparatus of claim 2, wherein the first surface of the polymeric laminate is arcuate.

7. The apparatus of claim 2, wherein an outer perimeter of the polymeric laminate extends outward of an outer perimeter of the first surface of the first portion of the locking mechanism.

8. The apparatus of claim 1, wherein the polymeric laminate includes one or more apertures extending through the polymeric laminate.

9. The apparatus of claim 8, wherein the one or more apertures align with one or more corresponding apertures formed in the first portion of the locking mechanism.

10. A replacement heart valve implant, comprising:
an expandable anchor member actuatable between a delivery configuration and a deployed configuration;
a plurality of locking mechanisms configured to maintain the expandable anchor member in the deployed configuration;
one or more valve leaflets attached to each of the plurality of locking mechanisms; and
at least one polymeric laminate corresponding to each locking mechanism, each polymeric laminate sized and configured for mounting between a first portion of the corresponding locking mechanism and the expandable anchor member.

11. The replacement heart valve implant of claim 10, wherein each locking mechanism is non-removably secured to the expandable anchor member.

12. The replacement heart valve implant of claim 10, wherein actuation of the expandable anchor member between the delivery configuration and the deployed configuration causes relative movement between the expandable anchor member and each locking mechanism.

13. The replacement heart valve implant of claim 10, wherein the expandable anchor member is formed with a braided configuration.

14. The replacement heart valve implant of claim 10, wherein each locking mechanism comprises a buckle member secured to the expandable anchor member and a translatable post member configured to engage the buckle member when the expandable anchor member is in the deployed configuration.

15. The replacement heart valve implant of claim 14, wherein the polymeric laminate prevents contact between the buckle member and the expandable anchor member.

16. A method of manufacturing an apparatus for reducing adhesive wear in a replacement heart valve implant, comprising:
stacking a plurality of layers of polyester fabric on a mandrel in a generally flat configuration;
spraying the plurality of layers with a polyurethane in the generally flat configuration, wherein the polyurethane impregnates the plurality of layers and adheres the plurality of layers together;
laser cutting a perimeter of the apparatus to separate the apparatus from the polyester fabric; and
forming the apparatus into a static three-dimensional shape corresponding to an exterior surface of a locking mechanism of the replacement heart valve implant.

17. The method of claim 16, wherein laser cutting the perimeter of the apparatus includes laser welding the plurality of layers together along the perimeter.

18. The method of claim 16, wherein forming the apparatus includes heat setting the apparatus using a mandrel or die set.

19. The method of claim 16, further comprising curing the plurality of layers after spraying the plurality of layers with polyurethane and before laser cutting.

20. The method of claim 16, further comprising securing the apparatus between the exterior surface of the locking mechanism and an expandable anchor member of the replacement heart valve implant, thereby preventing direct contact between the exterior surface of the locking mechanism and the expandable anchor member.
